(19) United States
(12) Patent Application Publication
(10) Pub. No.: US 2002/0169154 A1
(43) Pub. Date: Nov. 14, 2002

(54) Novel methods and compositions involving trk tyrosine kinase inhibitors and antineoplastic agents

(75) Inventors: Bruce A. Ruggeri, West Chester, PA (US); Susan E. Jones-Bolin, Thorndale, PA (US)

Correspondence Address: Robert T. Hrubiec, CEPHALON, INC., 145 Brandywine Parkway, West Chester, PA, 19380, US

(73) Assignee: Cephalon, Inc.

(21) Appl. No.: 10/115,407

(22) Filed: Apr. 3, 2002

Related U.S. Application Data
(60) Provisional application No. 60/281,366, filed on Apr. 4, 2001.

(51) Int. Cl. A61K 31/553

(52) U.S. Cl. 514/211.08

(57) ABSTRACT

Novel methods and compositions comprising antineoplastic agents and trk tyrosine kinase inhibitors are disclosed. In preferred embodiments, the antineoplastic agents comprise nucleoside analogs, and the trk tyrosine kinase inhibitors comprise indolocarbazoles and indenocarbazoles. The methods and compositions may be suitable for the treatment of cancer, particularly pancreatic cancer.

### FIELD OF THE INVENTION

The present invention relates to novel methods and compositions involving trk tyrosine kinase inhibitors and antineoplastic agents. More particularly, the present invention relates to novel methods and compositions involving trk tyrosine kinase inhibitors and antineoplastic agents for the treatment of cancer, including pancreatic cancer.

### BACKGROUND OF THE INVENTION

Broadly speaking, the main approaches to cancer treatment include radiation therapy, surgery and chemotherapy. Many patients undergoing these treatments, however, do not experience substantial, sustained, favorable responses.

Radiation and surgical approaches generally necessitate a complete or substantially complete removal or destruction of all cancer cells in order to render effective treatment. In practice, such results can rarely be achieved, and are extremely difficult where metastasis has occurred. Moreover, radiation therapy and surgery generally aid only in the local control of cancer and typically offer limited benefit for patient survival.

Chemotherapeutic agents have been developed that exhibit antitumor activity against a variety of solid tumors, but the prognosis and overall survival rate of cancer patients undergoing chemotherapy remains poor. Furthermore, the use of chemotherapeutic agents is often associated with systemic toxicity, which may result in severe side effects such as nausea, vomiting, mucositis, neutropenia, thrombocytopenia, and anorexia.

Chemotherapeutic agents include nucleoside analogs and trk tyrosine kinase inhibitors. Gemcitabine (2′,2′-difluorodeoxycytidine) is a nucleoside analog, the incorporation of which into DNA has been shown to prevent the elongation of nascent DNA strands. Gemcitabine has exhibited antitumor activity against a variety of tumors. See Lund; B., et al. J. Natl. Cancer Inst. 86:1530-1533, 1994; Hertel, L. W., et al. Cancer Res.50:4417-4422, 1990; Grindley, G. B., et al. Cancer Invest. 8:313-318, 1990. Treatment with gemcitabine, however, as with other chemotherapeutic agents, has not been shown to significantly enhance patient survival in most cases. See Rothenberg, M. L. et al. Ann. Oncol. 7:347-353, 1996.

Neurotrophins regulate growth, differentiation and survival of central and peripheral neurons. Neurotrophin growth factors bind to and activate cell surface receptors, trks, which exhibit tyrosine kinase activity. The binding of neurotrophin to a trk receptor leads to receptor oligomerization and tyrosine phosphorylation of specific intracellular substrates. A possible role for neurotrophins in the invasive and metastatic phenotype of specific tumor types, and the modulation of cell survival pathways in other types of cancer, has recently been recognized. See Ruggeri. B. A., et al. Current Med. Chem. 6:845-857, 1999. Furthermore, trk tyrosine kinase inhibitors have been shown to exhibit antitumor activity. See Miknyoczki, S. J., et al. Clin. Cancer Res. 5:2205-2212, 1999. The antitumor efficacy of trk tyrosine kinase inhibitors, however, is generally not significantly greater than that of other chemotherapeutic agents. Id. A need thus exists for improved cancer treatments that may desirably prolong the survival of cancer patients, preferably without producing severe side effects. The present invention is directed to these, as well as other important ends.

### SUMMARY OF THE INVENTION

The present invention is directed, in part, to novel methods and compositions for treating cancer. Specifically, in one embodiment, methods of treating cancer are provided that comprise administering to a patient an effective amount of a trk tyrosine kinase inhibitor in combination with an effective amount of an antineoplastic agent.

Another embodiment of the invention relates to pharmaceutical compositions comprising an effective amount of a trk tyrosine kinase inhibitor and an effective amount of an antineoplastic agent, together with a pharmaceutically acceptable carrier.

Yet another embodiment of the invention relates to pharmaceutical kits comprising one or more containers containing pharmaceutical dosage units comprising an effective amount of a trk tyrosine kinase inhibitor in combination with an effective amount of an antineoplastic agent.

In preferred embodiments, the methods, compositions, and kits of the present invention involve a trk tyrosine kinase inhibitor of the following formula (I):

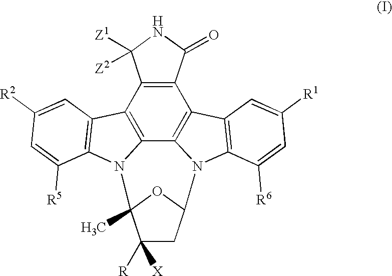

or a stereoisomer or pharmaceutically acceptable salt form thereof, wherein:

(a) when Z¹ and Z² are both hydrogen:

(1) R is selected from the group consisting of OH, O-n-alkyl of 1-6 carbons, and O-acyl of 2-6 carbons;

(2) X is selected from the group consisting of H; CONHC₆H₅ with the proviso that both R¹ and R² are not Br; CH₂Y wherein Y is:

OR⁷ wherein R⁷ is H or acyl of 2-5 carbons;

SOR⁸ wherein R8 is alkyl of 1-3 carbons, aryl, or a heterocyclic

group including a nitrogen atom;

NR⁹R¹⁰ wherein R⁹ and R10 are each independently H, alkyl of 1-3 carbons, Pro, Ser, Gly, Lys, or acyl of 2-5 carbons, with the proviso that only one of R⁹ and R¹⁰ is Pro, Ser, Gly, Lys or acyl;

SR¹⁶ wherein R¹⁶ is an aryl, alkyl of 1-3 carbons or a heterocyclic group that includes a nitrogen atom;

N₃; CO₂CH₃; S-Glc;

CONR¹¹R¹² wherein R¹¹ and R¹² are each independently H, alkyl of 1-6 carbons, C₆H₅, or hydroxyalkyl of 1-6 carbons, or R¹¹ and R¹² are combined to form —CH₂CH₂OCH₂CH₂—;

CO₂CH₃; CH═NNHCONH₂; CONHOH; CH═NOH;

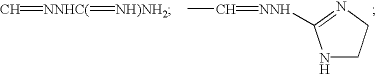

CH═NN(R¹⁷)₂ wherein R¹⁷ represents aryl;

CH₂NHCONHR¹⁸ wherein R¹⁸ is lower alkyl or aryl; or

X and R are combined together to form —CH₂NHCO₂—, —CH₂OC(CH₃)₂O—, ═O, or —CH₂N(CH₃)CO₂—;

(3) each of R¹, R², R⁵ and R⁶ is, independently, H or up to two of them are F; Cl; Br; I; NO₂; CN; OH; NHCONHR¹³ wherein R¹³ is C₆Hs or alkyl of 1-3 carbons with the proviso that only one of R¹, R², R⁵ and R⁶ is NHCONHR¹³; CH₂OR ; alkyl of 1-3 carbons;

CH₂OCONHR¹⁴; or NHCO₂R¹⁴; in which R¹⁴ is lower alkyl; CH(SC₆H₅)₂ or

CH(—SCH₂CH₂S—); or R¹ is CH₂S(O)ₚR²¹ where p=0 or 1, and R²¹ is aryl, alkyl of 1-3 carbons, a heterocyclic group that includes a nitrogen atom,

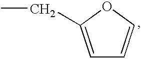

or CH₂CH₂N(CH₃)₂, and R², R5, and R are H; or R¹ is CH═NNR²R³, wherein R²² and R²³ are each independently H, alkyl of 1-3 carbons, C(═NH)NH₂, or a heterocyclic group that includes a nitrogen atom, or R²² and R²³ are combined together to form —(CH₂)₄—, —(CH₂CH₂OCH₂CH₂)—, or —(CH₂CH₂N(CH₃)CH₂CH₂)—, with the proviso that R²² and R²³ cannot both be H, and at least one of R²² or R²³ is H, except when both are alkyl, and R², R⁵ and R⁶are H;

and

(b) when Z¹ and Z² are both combined together to represent 0; X is CO₂CH₃; R is OH and R¹, R², R⁵ and R⁶ are each hydrogen.

In additional preferred embodiments, the present methods, compositions and kits involve an antineoplastic agent of the following formula (II):

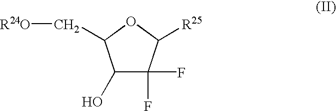

or a pharmaceutically acceptable salt form thereof, wherein:

R²⁴ is selected from H and —C(═O)—R28;

R²⁵ is a base defined by one of the following formulae:

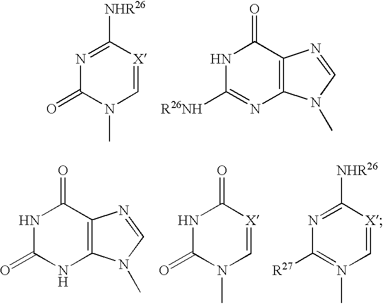

X′ is selected from N and C—R²⁷;

R²⁶ is selected from H. alkyl and —C(═O)—R²⁸;

R²⁷ is selected from H, alkyl, amino, bromo, fluoro, chloro and iodo; and

R²⁸ is selected from H and alkyl;

and a pharmaceutically acceptable carrier.

In other preferred embodiments, the present invention involves a trk kinase inhibitor of the following formula (III):

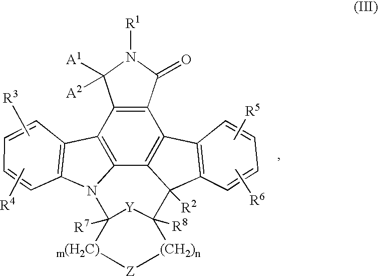

or a stereoisomer or pharmaceutically acceptable salt form thereof, wherein A¹, A², R¹—R⁸, Y, Z, m and n are as defined below.

These and other aspects of the invention will become more apparent from the following detailed description.

### BRIEF DESCRIPTION OF THE DRAWINGS

FIG. 1 illustrates Kaplan-Meier survival curves for nude mice orthotopically implanted with human pancreatic ductal adenocarcinoma (PDAC) xenografts and administered the compound of formula (I-a-i) and gemcitabine alone or in combination. The data indicate differences in survival (the number of days dosed) for untreated mice (U), vehicle treated mice (V), mice who received the compound of formula (I-aii) only (A), mice who received gemcitabine only (H), and mice who received a combination of the compound of formula (I-ai) and gemSitabine (D).

FIG. 2 illustrates Kaplan-Meier survival curves for nude mice orthotopically implanted with human pancreatic ductal adenocarcinoma (PDAC) xenografts and administered the compound of formula (m-a-i) and gemcitabine alone or in combination. The data indicate differences in survival (the number of days dosed) for untreated mice (U), vehicle treated mice (V), mice who received the compound of formula (I-a-i) only (A), mice who received gemcitabine only (H), and mice who received a combination of the compound of formula (I-a-ii) and gemcitabine (D).

### DETAILED DESCRIPTION OF THE INVENTION

As employed above and throughout the disclosure, the following terms, unless otherwise indicated, shall be understood to have the following meanings.

The core structures provided herein are presented by way of general guidance, and are not to be taken as limiting the scope of the invention. For example, certain cores indicate the presence of certain atoms for illustrative purposes. It will be appreciated that such atoms may bonded to additional groups, or may be further substituted without deviating from the spirit of the invention.

As used herein, “indolocarbazole” is intended to indicate a compound of the following formula:

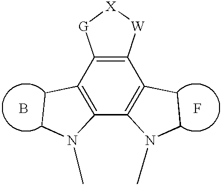

wherein at least one of G, X, or W is nitrogen and B and F are aryl or heteroaryl groups.

These compounds are intended to include, but are not limited to, structures in which the nitrogens of the carbazole and the indole form a cyclic bridged moiety:

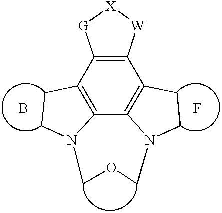

The indolocarbazoles and indenocarbazoles suitable for use in the methods and compositions of the present invention are stable compounds. As used herein “stable compound” or “stable structure” is meant to indicate a compound that is sufficiently robust to survive isolation to a useful degree of purity from a reaction mixture, and preferably capable of formulation into an efficacious therapeutic agent.

The indolocarbazoles and indenocarbazoles may be further substituted. As used herein, “substituted” is intended to indicate that one or more hydrogen atoms on the indicated atom is replaced with a selected group referred to herein as a “substituent”, provided that the substituted atom's valency is not exceeded, and that the substitution results in a stable compound.

As used herein, the term “alkyl” means a straight-chain, cyclic, or branched alkyl group having 1 to 8 carbon atoms, such as methyl, ethyl, propyl, isopropyl, butyl, isobutyl, sec-butyl, tert-butyl, pentyl, isoamyl, neopentyl, 1-ethylpropyl, hexyl, octyl, cyclopropyl, and cyclopentyl. The alkyl moiety of alkyl-containing groups, such as alkoxy, alkoxycarbonyl, and alkylaminocarbonyl groups, has the same meaning as alkyl defined above. Lower alkyl groups, which are preferred, are alkyl groups as defined above which contain 1 to 4 carbons.

Alkyl groups and alkyl moieties contained within substituent groups such as aralkyl, alkoxy, arylalkoxy, hydroxyalkoxy, alkoxy-alkoxy, hydroxy-alkylthio, alkoxy-alkylthio, alkylcarbonyloxy, hydroxyalkyl and acyloxy groups may be substituted or unsubstituted. A substituted alkyl group has 1 to 3 independently-selected substituents, preferably hydroxy, lower alkoxy, lower alkoxy-alkoxy, halogen, carboxyl, lower alkoxycarbonyl, nitro, amino, mono- or di-lower alkylamino, dioxolane, dioxane, dithiolane, dithione, furan, lactone, or lactam.

As used herein, the “acyl” moiety of acyl-containing groups such as acyloxy groups is intended to include a straight-chain, branched, or cyclic alkanoyl group having 1 to 6 carbon atoms, such as formyl, acetyl, propanoyl, butyryl, valeryl, pivaloyl or hexanoyl.

As used herein, the term “carbocyclic” refers to cyclic groups in which the ring portion is composed solely of carbon atoms. These include, but are not limited to, cyclopropyl, cyclobutyl, cyclopentyl, cyclohexl, cycloheptyl, cyclooctyl. The term “carbocyclic aromatic ring” is intended to include carbocyclic rings which are also aryl rings. The terms “heterocyclo” and “heterocyclic” refer to cyclic groups in which the ring portion includes at least one heteroatom such as O, N, or S. Heterocyclyl groups include heteroaryl and heteroalkyl groups.

As used herein the term “aryl” means an aromatic ring having 6 to 12 carbon atoms such as phenyl, biphenyl and naphthyl, with aromatic rings of 6 to 10 carbons being preferred. Preferred aryl groups include unsubstituted or substituted phenyl and naphthyl groups. The term “heteroaryl” as used herein denotes an aryl group in which one or more ring carbon atoms is replaced by a hetero (i.e., non-carbon) atom such as O, N or S. Preferred heteroaryl groups include pyridyl, pyrimidyl, pyrrolyl, furyl, thienyl, imidazolyl, triazolyl, tetrazolyl, quinolyl, isoquinolyl, benzoimidazolyl, thiazolyl, pyrazolyl, and benzothiazolyl groups. The term “heterocycloalkyl” denotes a cycloalkyl group in which , O one or more ring carbon atoms is replaced by hetero atoms such as O, N, or S.

As used herein, the term “aralkyl” (or “arylalkyl”) is intended to denote a group having from 7 to 15 carbons, consisting of an alkyl group that bears an aryl group. Examples of aralkyl groups include, but are not limited to, benzyl, phenethyl, benzhydryl and naphthylmethyl groups. Substituted aryl, substituted heterocyclic and substituted aralkyl groups each have 1 to 3 independently selected substituents that are preferably lower alkyl, hydroxy, lower alkoxy, carboxy, lower alkoxycarbonyl, nitro, amino, mono- or di-lower alkylamino, and halogen.

Preferred heterocyclic groups formed with a nitrogen atom include pyrrolidinyl, piperidinyl, piperidino, morpholinyl, morpholino, thiomorpholino, N-methylpiperazinyl, indolyl, isoindolyl, imidazole, imidazoline, oxazoline, oxazole, triazole, thiazoline, thiazole, isothiazole, thiadiazoles, triazines, isoxazole, oxindole, indoxyl, pyrazole, pyrazolone, pyrimidine, pyrazine, quinoline, iosquinoline, and tetrazole groups. Preferred heterocyclic groups formed with an oxygen atom include furan, tetrahydrofuran, pyran, benzofurans, isobenzofurans, and tetrahydropyran groups. Preferred heterocyclic groups formed with a sulfur atom include thiophene, thianaphthene, tetrahydrothiophene, tetrahydrothiapyran, and benzothiophenes.

As used herein, “hydroxyalkyl” groups are alkyl groups that have a hydroxyl group appended thereto. As used herein, “hydroxyalkoxy” groups are alkoxy groups that have a hydroxyl group appended thereto. As used herein, “halogen” refers to fluorine, chlorine, bromine and iodine.

As used herein, the term “amino acid” denotes a molecule containing both an amino group and a carboxyl group. Embodiments of amino acids include (t-amino acids; i.e., carboxylic acids of general formula HOOC—CH(NH₂)-(side chain). Side chains of amino acids include naturally occurring and non-naturally occurring moieties. Non-naturally occurring (i.e., unnatural) amino acid side chains are moieties that are used in place of naturally occurring amino acid side chains in, for example, amino acid analogs. See, for example, Lehninger, Biochemistry, 2nd Ed., Worth Publishers, Inc, 1975, pp. 73-75, incorporated by reference herein. As used herein “Pro” denotes proline, “Ser” denotes Serine, “Gly” denotes glycine, and “Lys” denotes lysine. In certain embodiments, substituent groups for the compounds described herein include the residue of an amino acid after removal of the hydroxyl moiety of the carboxyl group thereof; i.e., groups of formula —C(═O)CH(NH₂)-(side chain).

The compounds of the invention may be present in various forms as will be appreciated by the skilled artisan. Such forms include, but are not limited to, pharmaceutically acceptable salts, prodrugs, polymorphs, stereoisomers, and the like. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media like ether, ethyl acetate, ethanol, isopropanol, or acetonitrile are preferred. Lists of suitable salts are found in Remington's Pharmaceutical Sciences, 17th Ed., Mack Pub. Co., Easton, Pa., 1985, p. 1418, the disclosure of which is hereby incorporated by reference.

“Effective amount” refers to an amount of a compound as described herein that may be therapeutically effective to inhibit, prevent or treat the symptoms of particular disease or disorder. Such diseases and disorders include, but are not limited to, those pathological conditions associated with uncontrolled cell growth. Thus, for example, the term “effective amount”, when used in connection with the compounds of the present invention refers to the treatment and/or prevention of uncontrolled cell growth.

“In combination with”, “combination therapy” and “combination products” refer, in certain embodiments, to the concurrent administration to a patient of a trk tyrosine kinase inhibitor and an antineoplastic agent, including, for example, the compounds of formulas (I) and (II). When administered in combination, each component may be administered at the same time or sequentially in any order at different points in time. Thus, each component may be administered separately but sufficiently closely in time so as to provide the desired therapeutic effect. “Dosage unit” refers to physically discrete units suited as unitary dosages for the particular individual to be treated. Each unit may contain a predetermined quantity of active compound(s) calculated to produce the desired therapeutic effect(s) in association with the required pharmaceutical carrier. The specification for the dosage unit forms of the invention may be dictated by (a) the unique characteristics of the active compound(s) and the particular therapeutic effect(s) to be achieved, and (b) the limitations inherent in the art of compounding such active compound(s).

“Patient” refers to animals, including mammals, preferably humans.

The present invention provides methods and compositions that may be useful for the treatment of cancer. More specifically, the present invention is directed to methods and pharmaceutical compositions comprising a combination of a trk tyrosine kinase inhibitor and an antineoplastic agent. As noted above, antineoplastic agents have been used in systemic chemotherapy and have shown antitumor activity against a variety of solid tumors, but the prognosis and overall survival of cancer patients undergoing chemotherapy remains poor. In addition, anti-tumor efficacy has been demonstrated for various trk tyrosine kinase inhibitors. Chemotherapy with either antineoplastic agents, or trk tyrosine kinase inhibitors, however, has not provided a desirable increase in the survival of patients. Surprisingly, it has been unexpectedly discovered that treatment with a combination of a trk tyrosine kinase inhibitor and an antineoplastic agent, according to the methods and compositions of the present invention, may desirably and advantageously prolong survival. The enhanced survival that may be achieved through combination therapy with a trk tyrosine kinase inhibitor and an antineoplastic agent, as described herein, may effectively be greater than a simple additive effect of the two agents, when administered separately.

The present invention is directed to combination therapy for the treatment of cancer. More specifically, the invention is directed to methods for the treatment of cancer that involve the administration of a combination of a trk tyrosine kinase inhibitor and an antineoplastic agent. The invention is further directed to compositions comprising a trk tyrosine kinase inhibitor and an antineoplastic agent. The present combination methods and compositions may be used to treat patients with cancer, and preferably to significantly increase the survival of cancer patients, relative to presently-available treatments.

The methods and compositions of the present invention may be used to treat a wide variety of cancers, including, for example, carcinomas of the pancreas, prostate, breast, thyroid, colon, and lung; malignant melanomas; glioblastomas; neuroectodermal-derived tumors including Wilm's tumor, neuroblastomas, and medulloblastomas; and leukemias including, but not limited to, acute myelogenous leukemia (“ALM”), chronic myelogenous leukemia (“CLM”), acute lymphocytic leukemia (“ALL”), and chronic lymphocytic leukemia (“CLL”). Of these, the present methods and compositions may preferably be used to treat carcinomas of the prostate and pancreas, with carcinomas of the pancreas being especially suitable for treatment. Exemplary of carcinomas of the pancreas that may be treated with the present methods and compositions is pancreatic ductal adenocarcinoma (PDAC).

Other carcinomas that may be treated with the methods and compositions of the present invention, in addition to those exemplified above, would be readily apparent to one of ordinary skill in the art, once armed with the teachings of the present disclosure.

As noted above, the methods and compositions of the present invention involve a trk tyrosine kinase inhibitor. A wide variety of trk tyrosine kinase inhibitors are available and may be suitable for use in the methods and compositions of the present invention. In preferred form, the trk tyrosine kinase inhibitor is an indolocarbazole. In certain preferred embodiments, the trk tyrosine kinase inhibitor is a compound of the following formula (I):

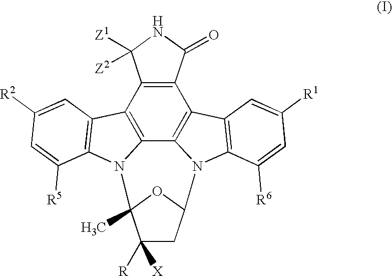

or a stereoisomer or pharmaceutically acceptable salt form thereof, wherein:

(a) when Z¹ and Z are both hydrogen:

(1) R is selected from the group consisting of OH, O-n-alkyl of 1-6 carbons, and O-acyl of 2-6 carbons;

(2) X is selected from the group consisting of H; CONHC₆H₅ with the proviso that both R¹ and R² are not Br; CH₂Y wherein Y is:

OR⁷ wherein R⁷ is H or acyl of 2-5 carbons;

SOR⁸ wherein R is alkyl of 1-3 carbons, aryl, or a heterocyclic group including a nitrogen atom;

NR⁹R¹⁰ wherein R⁹ and R¹⁰ are each independently H, alkyl of 1-3 carbons, Pro, Ser, Gly, Lys, or acyl of 2-5 carbons, with the proviso that only one of R⁹ and R¹⁰ is Pro, Ser, Gly, Lys or acyl;

SR¹⁶ wherein R¹⁶ is an aryl, alkyl of 1-3 carbons or a heterocyclic group that includes a nitrogen atom;

N₃; CO₂CH₃; S-Glc;

CONR¹¹R¹² wherein R¹¹ and R¹² are each independently H, alkyl of 1-6 carbons, CrH₅, or hydroxyalkyl of 1-6 carbons, or R¹ and R¹² are combined to form —CH₂CH₂OCH₂CH₂—; CO₂CH₃; CH═NNHCONH₂; CONHOH; CH═NOH;

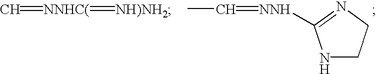

CH═NN(R¹⁷)₂ wherein R¹⁷ represents aryl; CH₂NHCONHR¹⁵ wherein R¹⁸ is lower alkyl or aryl; or

X and R are combined together to form —CH₂NHCO₂—, —CH₂OC(CH₃)₂O—, ═O, or —CH₂N(CH₃)CO₂—;

(3) each of R¹, R², R¹and R⁶ is, independently, H or up to two of them are F; Cl; Br; I; NO₂; CN; OH; NHCONHR¹³ wherein R¹³ is C₆H₅ or alkyl of 1-3 carbons with the proviso that only one of R¹, R², R⁵ and R⁶ is NHCONHR¹³; CH₂OR¹³; alkyl of 1-3 carbons; CH₂OCONHR¹⁴; or NHCO₂R⁴; in which R¹⁴ is lower alkyl; CH(SC₆HS)₂ or

CH(—SCH₂CH₂S—); or R¹ is CH₂S(O)ₚR²¹ where p=0 or 1, and R²¹ is aryl, alkyl of 1-3 carbons, a heterocyclic group that includes a nitrogen atom,

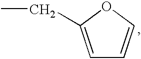

or CH₂CH₂N(CH₃)₂, and R², R⁵, and R⁶ are H; or R¹ is CH═NNR²²R²³, wherein R²² and R²³ are each independently H, alkyl of 1-3 carbons, C(═NH)NH₂, or a heterocyclic group that includes a nitrogen atom, or R²² and R²³ are combined together to form —(CH₂)₄—, —(CH₂CH₂OCH₂CH₂)—, or —(CH₂CH₂N(CH₃)CH₂CH₂)—, with the proviso that R²² and R²³ cannot both be H, and at least one of R²² or R²³ is H, except when both are alkyl, and R², R⁵ and R⁶ are H;

and

(b) when Z¹ and Z² are both combined together to represent 0; X is CO₂CH₃; R is OH and R¹, R², R⁵ and R⁶ are each hydrogen.

In particularly preferred embodiments, the compound of formula I has the following formula I-a:

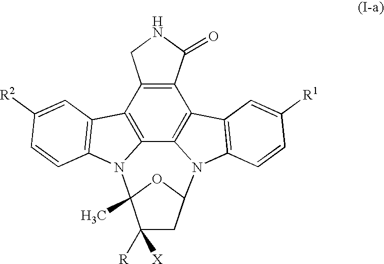

where R, X, R¹and R² are as previously described.

In preferred form, in the compounds of formula I-a, R¹and R² are selected from H, alkyl, Cl, Br, CH₂OH, CH₂SOCH₂CH₃, NHCONHC6H₅, CH₂SCH₂CH₃, CH₂SC₆H₅, NIHCO₂CH₃, CH₂OC(═O)NHCH₂CH₃, CH═NNH, and CH₂OCH₂CH₃; R is selected from OH and OCH₃; and X is selected from OH, CH₂OH, and CO₂alkyl.

In even more preferred embodiments, the compound of formula I-a is selected from:

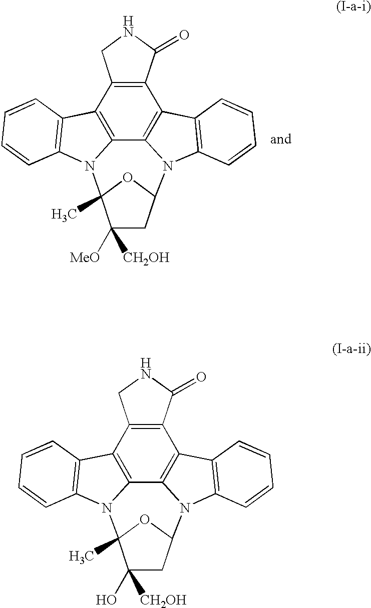

In still more preferred embodiments, the compound of formula (I-a) is:

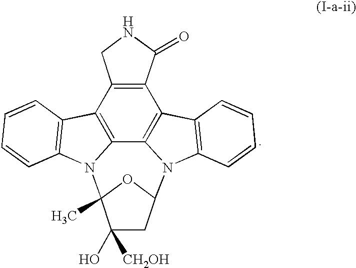

In another preferred form, the trk tyrosine kinase inhibitor is an indenocarbazole. In certain preferred embodiments, the trk tyrosine kinase inhibitor is a compound of the following formula (III):

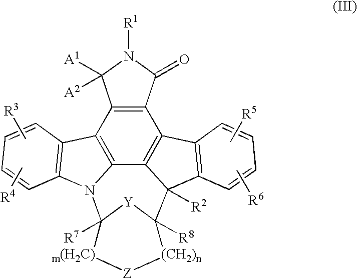

or a stereoisomer or pharmaceutically acceptable salt form thereof, wherein:

R¹ is selected from the group consisting of:

a) H, substituted or unsubstituted C₁₋₄ alkyl, substituted or unsubstituted aryl, substituted or unsubstituted arylalkyl, substituted or unsubstituted heteroaryl, or substituted or unsubstituted heteroarylalkyl;

b) —C(═O)R⁹, where R⁹ is selected from the group consisting of H, C₁₋₄ alkyl, aryl and heteroaryl;

c) —OR¹⁰O, where R¹⁰ is selected from the group consisting of H and C₁₋₄ alkyl;

d) —C(═O)NH₂, —NR¹¹R¹², —(CH₂)ₚNR¹¹R¹², —(CH₂)ₚOR , —O(CU²)ₚOR and —O(CH₂)ₚNR¹¹R¹², wherein p is from 1 to 4; and wherein either

1) R¹¹ and R¹² are each independently selected from the group consisting of H and C₁₋₄ alkyl; or

2) R¹¹ and R¹² together form a linking group of the formula —(CH₂)₂—X¹—(CH₂)₂—, wherein X¹ is selected from the group consisting of —O—, —S—, and —CH₂—;

R² is selected from the group consisting of H, C₁₋₄ alkyl, —OH, and C₁₋₄ alkoxy;

R³, R⁴, R⁵ and R⁶ are each independently selected from the group consisting of:

a) H, aryl, heteroaryl, F, Cl, Br, I, —CN, CF₃, —NO₂, —OH, —OR , —O(CH₂)ₚNR¹¹R¹²—OC(═O)R⁹, —OC(═O)NR²R⁷, —OC(═O)NR¹¹R¹², —O(CH₂)ₚOR¹⁰, —CH₂OR¹⁰, —NR¹¹R¹², —NR¹⁰S(═O)₂R⁹, NR¹⁰C(═O)R⁹;

b) —CH₂OR¹⁴, wherein R¹⁴ is the residue of an amino acid after the hydroxyl group of the carboxyl group is removed;

c) —NROC(═O)NR¹¹R¹², —CO₂R², —C(═O)R², —C(═O)NR¹¹R¹², —CH═NOR², —CH═NR⁹, —(CH₂)ₚNR¹¹R¹², —(CH₂)ₚNHR¹⁴, or —CH═NNR²R^{2A} wherein R^{2A} is the same as R²;

d) —S(O)_{y}R²—(CH₂)ₚS(O)_{y}R⁹, —CH₂S(O)_{y}R¹⁴ wherein y is 0, 1 or 2;

e) C₁₋₈ alkyl, C₂₋₈ alkenyl, C₂₋₈ alkynyl, wherein each alkyl, alkenyl, or alkynyl group may substituted with 1 to 3 groups selected from the group consisting of:

C₆₋₁₀ arylalkyl, heteroaryl, arylalkoxy, heterocycloalkoxy, hydroxyalkoxy, alkyloxy-alkoxy, hydroxyalkylthio, alkoxy-alkylthio, F, Cl, Br, I, —CN, —NO₂, —OH, —OR⁹, —X²(CH₂),NR IR¹, —X²(CH₂)ₚC(═O)NR¹¹R¹², —X²(CH₂)ₚOC(═O)NR¹¹R¹², —X² (CH₂)ₚCO₂R⁹, —X²(CH₂)ₚS(O)_{y}R⁹, —X²(CH₂)ₚNR¹¹C(═O)NR¹¹R¹², —OC(═O)R⁹, _OCONHR², —O-tetrahydropyranyl, —NR¹¹R¹², —NR¹⁰C(═O)R⁹, —NR¹⁰CO₂R⁹, —NR¹⁰C(═O)NR¹¹R², —NHC(═NH)NH₂, NR¹⁰S(O)₂R⁹, —S(O)_{y}R⁹, —CO₂R², —C(═O)NR¹¹R¹², —C(═O)R², —CH₂OR¹⁰, —CH═NNR²R^{2A}, —CH═NOR², —CH═NR⁹, —CH═NNHCH(N═NH)NH₂, —S(═O)₂NR²R^{2A}, —P(═O)(OR¹⁰)₂, —OR¹⁴, and a monosaccharide having from 5 to 7 carbons wherein each hydroxyl group of the monosaccharide is independently either unsubstituted or is replaced by H, C₁₋₄ alkyl, alkylcarbonyloxy having from 2 to 5 carbons, or C₁₋₄ alkoxy;

wherein X² is O, S, or NRO;

R⁷ and R⁸ are each independently selected from the group consisting of H, C₁₋₄ alkyl, C₁₋₄ alkoxy, substituted or unsubstituted C₆₋₁₀ arylalkyl, substituted or unsubstituted heteroarylalkyl, —(CH₂)ₚOR¹⁰, —(CH₂)ₚOC(═O)NR¹¹R¹², and —(CH₂)ₚNR¹¹R¹²;

or R⁷ and R⁸ together form a linking group of the formula —CH₂—X³—CH₂—; wherein X³ is X² or a bond;

m and n are each independently 0, 1, or 2;

Y is selected from the group consisting of —O—, —S—, —N(R¹⁰)—, —N⁺(O⁻)(R¹⁰)—, —N(OR¹⁰)—, and —CH₂—;

Z is selected from the group consisting of a bond, —O—, —CH═CH—, —S—, —C(═O)—, —CH(OR¹⁰)—, —N(R¹⁰)—, —N(OR¹⁰)—, CH(NR¹¹R¹²)—, —C(═O)N(R¹⁷)—, —N(R¹⁷)C(═O)—, —N(S(O)_{y}R⁹)—, —N(S(O)_{y}NR¹¹R¹²)—, —N(C(═O)R¹⁷)—, —C(R¹⁵R¹⁶)—, —N⁺(O⁻)(R¹⁰)—, —CH(OH)—CH(OH)—, and —CH(O(C═O)R⁹)CH(OC(═O)R⁹A)—;

wherein

R^{9A} is the same as R⁹;

R¹⁵ and R¹⁶ are independently selected from the group consisting of H, —OH, —C(═O)R¹⁰, —O(C═O)R⁹, hydroxyalkyl, and —CO₂R¹⁰;

R¹⁷ is selected from the group consisting of H, C₁₋₆ alkyl, aryl, and heteroaryl;

A¹ and A² are selected from the group consisting of H, H; H, OR²; H, —SR²; H, —N(R²)₂; and a group wherein A¹ and A² together form a moiety selected from the group consisting of ═O, ═S, and ═NR². In particularly preferred embodiments, the compound of formula (M) has the following formula (III-a):

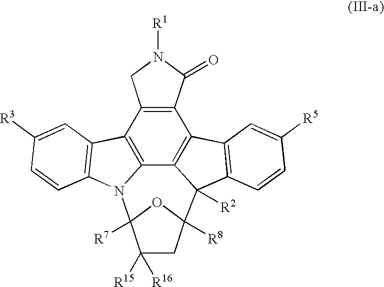

or a stereoisomer or pharmaceutically acceptable salt form thereof, wherein:

R¹ is H or substituted or unsubstituted C₁₋₄ alkyl;

R² is selected from the group consisting of H, C₁₋₄ alkyl, —OH, and C₁₋₄ alkoxy;

R³ and R⁵ are each independently selected from the group consisting of:

a) H, aryl, heteroaryl, F, Cl, Br, I, —CN, CF₃, —NO₂, —OH, —OR⁹, —O(CH₂)ₚNR¹¹R¹², —OC(═O)R⁹, —OC(═O)NR¹¹R¹², —OC(═O)NR¹¹R¹², —O(CH₂)ₚOR¹⁰, —CH₂OR¹⁰, —NR¹¹R¹², —NR¹⁰S(═O)₂R⁹, NR¹⁰C(═O)R⁹;

b) —CH₂OR¹⁴, wherein R¹⁴ is the residue of an amino acid after the hydroxyl group of the carboxyl group is removed;

c) —NR¹⁰C(═O)NR¹¹R¹², —CO₂R², —C(═O)R², —C(═O)NR¹¹R¹², —CH═NOR², —CH═NR⁹, —(CH₂)ₚNR¹¹R¹², —(CH₂)NH⁴, or —CH═NNR²R^{2A}; wherein R^{2A} is the same as R²;

d) —S(O)_{y}R²—(CH₂)ₚS(O)_{y}R⁹, —CH₂S(O)_{y}R¹⁴ wherein y is 0, 1 or 2;

e) C₁₋₈ alkyl, C₂₋₈ alkenyl, C₂₋₈ alkynyl, wherein each alkyl, alkenyl, or alkynyl group may substituted with 1 to 3 groups selected from the group consisting of:

C₆₋₁₀ arylalkyl, heteroaryl, arylalkoxy, heterocycloalkoxy, hydroxyalkoxy, alkyloxy-alkoxy, hydroxyalkylthio, alkoxyalkylthio, F, Cl, Br, I, —CN, —NO₂, —OH, —OR⁹, —X²(CH₂)ₚNR¹R , —X²(CH₂)ₚC(═O)NR¹¹R¹², —X²(CH₂)ₚOC(═O)NR¹¹R¹², —X²(CH₂)ₚCO₂R⁹, —X²(CH₂)ₚS(O)_{y}R⁹, —X²(CH₂)ₚNR¹⁰C(═O)NR¹¹R¹², —OC(═O)R , —OCONHR², —O-tetrahydropyranyl, —NR¹¹R¹², —NR¹⁰C(═O)R⁹, —NR¹⁰CO₂R⁹, NR¹⁰C.(═O)NR¹¹R¹²—NHC(═NH)NH₂, NR¹⁰S(O)₂R⁹, —S(O)_{y}R⁹, CO₂R²—C(═O)NR¹¹R¹², —C(═O)R², —CH₂OR¹⁰, —CH═NNR²R^{2A}, —CH═NOR², —CH═NR⁹, —CH═NNHCH(N═NH)NH₂, —S(═O)₂NR²R^{2A}, —P(═O)(OR¹⁰)₂, —OR¹⁴, and a monosaccharide having from 5 to 7 carbons wherein each hydroxyl group of the monosaccharide is independently either unsubstituted or is replaced by H, C₁₋₄ alkyl, alkylcarbonyloxy having from 2 to 5 carbons, or C₁₋₄ alkoxy; wherein

X² is O, S, or NR¹⁰;

R⁹ and R¹⁰ are each independently H or C₁₋₄ alkyl;

R¹¹ and R¹² are each independently H or C₁₋₄ alkyl; or R¹¹ and R¹² together form a linking group of the formula —(CH₂)₂—X¹—(CH₂)₂—, wherein X¹ is selected from the group consisting of —O—, —S—, and —CH₂—; and

p is from 1 to 4,

R⁷ and R⁸ are each independently H or substituted or unsubstituted C₁₋₄ alkyl; R¹⁵ and R¹⁶ are independently selected from the group consisting of H, —OH, —C(═O)R¹⁰, —O(C═O)R⁹, hydroxyalkyl, and —CO₂R¹⁰.

In a preferred embodiment, the compound of formula (III-a) is:

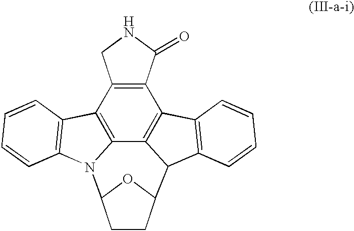

Other trk tyrosine kinase inhibitors that may be employed in the methods and compositions of the present invention, in addition to those exemplified above, would be readily apparent to one of ordinary skill in the art, once armed with the teachings of the present disclosure.

As noted above, the methods and compositions of the present invention further involve an antineoplastic agent. A wide variety of antineoplastic agents are available and may be suitable for use in the methods and compositions of the present invention. In preferred embodiments, the antineoplastic agent is selected from fluoropyrimidines, pyrimidine nucleosides and purines. In more preferred embodiments, the fluoropyrimidine is selected from 5-fluorouracil and ftorafur, the pyrimidine nucleoside is selected from gemcitabine, 5-azacytidine and cytosine arabinoside, and the purine is 6-thioguanine.

In preferred embodiments, the antineoplastic agent is a compound of the following formula (II):

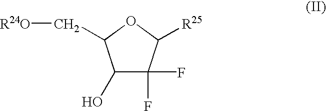

or a pharmaceutically acceptable salt form thereof, wherein:

R²⁴ is selected from H and —C(═O)—R²⁸;

R²⁵ is a base defined by one of the following formulae:

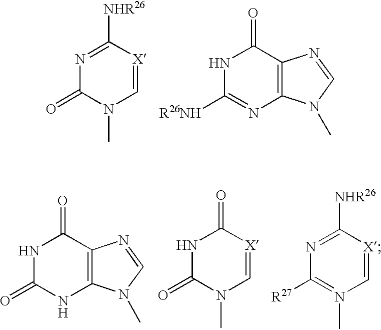

X′ is selected from N and C—R²⁷;

R²⁶ is selected from H, alkyl and —C(═O)—R²⁸;

R²⁷ is selected from H, alkyl, amino, bromo, fluoro, chloro and iodo; and

R²⁸ is selected from H and alkyl;

and a pharmaceutically acceptable carrier.

In particularly preferred embodiments, R²⁵ of formula (II) is a base of the following formula

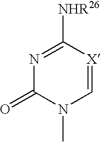

where X′ and R²⁶ are as previously described.

In preferred form, X′ of the above formula is C—R²⁷. In more preferred embodiments, R²⁴, R²⁶, R²⁷ and R²⁸ are H. An even more preferred antineoplastic agent for use in the present methods and compositions is gemcitabine, i.e., the compound of the following formula:

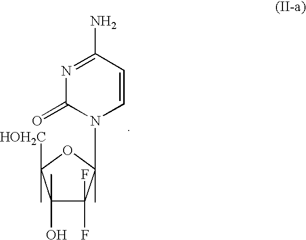

Thus, in particularly preferred form, the methods and compositions of the present invention involve a trk tyrosine kinase inhibitor of the following formula (I-a-ii):

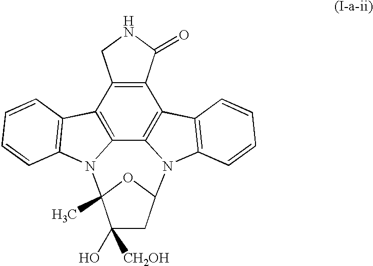

and an antineoplastic agent of the following formula (II-a):

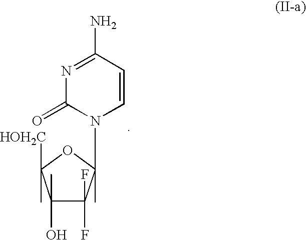

Thus, in another particularly preferred form, the methods and compositions of the present invention involve a trk tyrosine kinase inhibitor of the following formula (III-a-i):

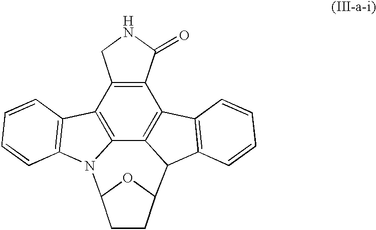

and an antineoplastic agent of the following formula (11-a):

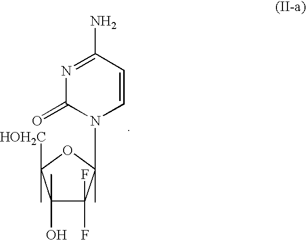

In more preferred embodiments, the methods and compositions of the present invention involve a trk tyrosine kinase inhibitor of formula (I-a-ii) and an antineoplastic agent of formula (11-a) for the treatment of pancreatic cancer, and particularly for the treatment of pancreatic ductal adenocarcinoma.

In other more preferred embodiments, the methods and compositions of the present invention involve a trk tyrosine kinase inhibitor of formula (II-a) and an antineoplastic agent of formula (II-a) for the treatment of pancreatic cancer, and particularly for the treatment of pancreatic ductal adenocarcinoma.

In certain preferred embodiments of the invention, the antineoplastic agent is administered to the patient in a substantial excess relative to the trk tyrosine kinase inhibitor. The term “substantial excess”, as used herein, means that the antineoplastic agent is preferably administered in an amount of at least twice as much, on a weight basis, relative to the trk tyrosine kinase inhibitor. In more preferred embodiments, the antineoplastic agent and the trk tyrosine kinase inhibitor are administered to the patient in a weight ratio of from about 2:1 to about 50:1, more preferably from about 4:1 to about 20:1, and even more preferably from about 8:1 to about 16:1.

By way of general guidance, the daily dosage of the antineoplastic agent, when used for the indicated effects, will range from about 1 mg/kg to about 300 mg/kg (and all combinations and subcombinations of ranges and specific dosages therein), preferably from about 10 mg/kg to about 200 mg/kg, and more preferably from about 50 mg/kg to about 150 mg/kg. The dosage units for the antineoplastic agent of the method may alternatively be expressed as mg/mm²; accordingly, in a preferred embodiment the antineoplastic agent may be administered in an amount which ranges from about 30 mg/mm² to about 900 mg/mm² (and all combinations and subcombinations of ranges and specific dosages therein), and more preferably from about 150 mg/mm² to about 600 mg/mm², and even more preferably from about 210 mg/mm² to about 450 mg/mm².

Also by way of general guidance, the daily dosage of the trk tyrosine kinase inhibitor may range from about 1 mg/kg to about 200 mg/kg (and all combinations and subcombinations of ranges and specific dosages therein), preferably from about 2 mg/kg to about 100 mg/kg, and more preferably from about 5 mg/kg to about 50 mg/kg.

In connection with the compositions of the present invention, the antineoplastic agent is preferably present in the compositions in a substantial excess relative to the trk tyrosine kinase inhibitor. In certain preferred embodiments, the antineoplastic agent and the trk tyrosine kinase inhibitor are present in the compositions in a weight ratio of from about 2:1 to about 50: 1, more preferably from about 4:1 to about 20:1, and most preferably from about 8:1 to about 16:1.

In certain preferred embodiments, the compositions are in a single dosage unit form.

Another embodiment of the invention includes pharmaceutical kits comprising one or more containers containing pharmaceutical dosage units comprising an effective amount of a trk tyrosine kinase inhibitor in combination with an effective amount of an antineoplastic agent.

In certain preferred embodiments, the kits further comprising conventional pharmaceutical kit components.

The compounds employed in the methods of the present invention may exist in prodrug form. As used herein, “prodrug” is intended to include any covalently bonded carriers which release the active parent drug, for example, as according to formulas (I) or (II) or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example formula (I), may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and arylalkyl esters such as methyl, ethyl, propyl, iso-propyl, butyl, isobutyl, sec-butyl, tert-butyl, cyclopropyl, phenyl, benzyl, and phenethyl esters, and the like.

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

It will be appreciated that the compounds of the present invention may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Preferred protecting groups include the benzyloxycarbonyl (Cbz; Z) group and the tert-butyloxycarbonyl (13oc) group. Other preferred protecting groups according to the invention may be found in Greene, T. W. and Wuts, P. G. M., Protective Groups in Organic Synthesis 2nd Ed., Wiley & Sons, 1991.

Indolocarbazoles may be synthesized by methods taught, for example, in U.S. Pat. Nos. 4,923,986; 4,877,776; 5,093,330; 5,461,146; 5,468,872; 5,621,100; 5,621,101; 5,516,771; and 5,599,808; and PCT publication Nos. WO 93/08809 and WO 97/46565, the discloses of which are hereby incorporated herein by reference in their entireties. Additional methods of preparation are set forth in Moody et al., J. Org. Chem. 57:2105-2114, 1992, also incorporated herein by reference.

Indenocarbazoles may be synthesized by methods taught, for example, in U.S. Pat. No. 6,127,401, the disclosure of which is hereby incorporated herein by reference in its entirety.

Antineoplastic agents useful in the invention may be synthesized by methods known in the art. The compounds of formula (II) may be synthesized by the methods taught, for example, in U.S. Pat. Nos. 4,808,614 and 5,464,826, the disclosures of which are hereby incorporated herein by reference in their entireties.

The compounds employed in the methods of the present invention including, for example, antineoplastic agents and trk tyrosine kinase inhibitors, may be administered by any means that results in the contact of the active agents with the agents' site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

The compounds are preferably combined with a pharmaceutical carrier selected on the basis of the chosen route of administration and standard pharmaceutical practice as described, for example, in Remington's Pharmaceutical Sciences, 17th Ed., Mack Pub. Co., Easton, Pa., 1985, the disclosures of which are hereby incorporated herein by reference, in their entirety.

Compounds of the present invention can be administered to a mammalian host in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

The active compound may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained. Preferred compositions or preparations according to the present invention may be prepared so that an oral dosage unit form contains from about 0.1 to about 1000 mg of active compound.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The active compound may also be administered parenterally or intraperitoneally. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze drying technique which yield a powder of the active ingredient, plus any additional desired ingredient from the previously sterile-filtered solution thereof.

The therapeutic compounds of this invention may be administered to a patient alone or in combination with a pharmaceutically acceptable carrier. As noted above, the relative proportions of active ingredient and carrier may be determined, for example, by the solubility and chemical nature of the compounds, chosen route of administration and standard pharmaceutical practice.

The dosage of the compounds of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached. Generally speaking, oral administration may require higher dosages.

The combination products of the invention, such as pharmaceutical compositions comprising an antineoplastic agent and a trk tyrosine kinase inhibitor, may be in any dosage form, such as those described herein, and can also be administered in various ways, as described herein. In a preferred embodiment, the combination products of the invention are formulated together, in a single dosage form (that is, combined together in one capsule, tablet, powder, or liquid, etc.). When the combination products are not formulated together in a single dosage form, the antineoplastic agent and the trk tyrosine kinase inhibitor may be administered at the same time (that is, together), or at different times. When not administered at the same time, preferably the administration of the antineoplastic agent and the trk tyrosine kinase inhibitor occurs less than about one hour apart, more preferably less than about 30 minutes apart, even more preferably less than about 15 minutes apart, and still more preferably less than about 5 minutes apart. Alternatively, the antineoplastic agent and the trk tyrosine kinase inhibitor may be administered at different times that occur one or more hours, or even days, apart.

Preferably, administration of the combination products of the invention is oral, although other routes of administration, as described above, are contemplated to be within the scope of the present invention. Although it is preferable that the antineoplastic agent and the trk tyrosine kinase inhibitor are both administered in the same fashion (that is, for example, both orally), if desired, they may each be administered in different fashions (that is, for example, one component of the combination product may be administered orally, and another component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

The proper dosage of the combination products of this invention will be readily ascertainable by one skilled in the art, once armed with the present disclosure. With regard to a typical dosage form of this type of combination product, such as a tablet, the antineoplastic agent generally may be present in an amount which ranges from about 10 to about 200 milligrams (and all combinations and subcombinations of ranges and specific dosages therein), and the trk tyrosine kinase inhibitor generally may be present in an amount which ranges from about 0.5 to about 100 milligrams (and all combinations and subcombinations of ranges and specific dosages therein).

Particularly when provided as a single dosage form, the potential exists for a chemical interaction between the combined active ingredients (for example, the antineoplastic agent and trk tyrosine kinase inhibitor). For this reason, the preferred dosage forms of the combination products of this invention are formulated such that although the active ingredients are combined in a single dosage form, the physical contact between the active ingredients is minimized (that is, reduced).

In order to minimize contact, one embodiment of the invention where the product is orally administered provides for a combination product wherein one active ingredient is enteric coated. By enteric coating one or more of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material which effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

These as well as other ways of minimizing contact between the components of combination products of the present invention, whether administered in a single dosage form or administered in separate forms but at the same time by the same manner, will be readily apparent to those skilled in the art, once armed with the present disclosure.

Pharmaceutical kits useful in, for example, the treatment of cancer, which comprise a therapeutically effective amount of an antineoplastic agent along with a therapeutically effective amount of a trk tyrosine kinase inhibitor, in one or more sterile containers, are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL two-part container (available from Abbott Labs, Chicago, Ill.), as desired. The antineoplastic agent and the trk tyrosine kinase inhibitor may be separate, or combined into a single dosage form as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

The materials, methods, and examples presented herein are intended to be illustrative, and not intended to limit the scope of the invention. All publications, patent applications, patents, and other references mentioned herein are incorporated by reference in their entirety. Unless otherwise defined, all technical and scientific terms are intended to have their art-recognized meanings.

### EXAMPLES

The invention is further demonstrated in the following examples. All of the examples are actual examples. The examples are for purposes of illustration and are not intended to limit the scope of the present invention.

### Example 1

### Formulations

The compound of formula (I-a-ii) was formulated at 3.44 mg/mil in a vehicle of 40% polyethylene glycol (PEG) 1000 (Spectrum, Los Angeles, Calif.), 10% povidone C30 (ISP, Boudbrook, N.J.), and 2% benzyl alcohol (Specturm, Los Angeles, Calif.) in sterile injectable water.

Gemcitabine HCl (Gemzar) was obtained from Eli Lilly and Co. (Indianapolis, Ind.) as a sterile, lyophilized powder formulated with mannitol and sodium acetate, and resuspended to a concentration of 1.0 mg/ml with 0.9% sodium chloride, pH 7.0. Solutions of gemcitabine were freshly prepared for each day of dosing and filter sterilized prior to administration.

### Example 2

### Tumor Xenograft Preparation

Human pancreatic ductal adenocarcinoma (PDAC) xenografts were obtained from the left flanks of nude mice that had been injected with 2×10⁶ Colo-357 human pancreatic carcinoma cells. Colo-357 human PDAC cells were obtained from the American Type Culture Collection (ATCC) and grown to subconfluency in Minimal Essential Medium supplemented with 10% fetal bovine serum and 1% penicillin/streptomycin. Cells were MAP and mycoplasma tested by a commercial laboratory (Bio Reliance Corp.) prior to use. Xenograft tumors were harvested and carefully sectioned into 2×2 mm² fragments in a laminar flow hood using sterile technique (PNAS 89:5645-49, 1992; Cancer Res. 55:4670-75, 1995). Netrotic areas of the tumors were removed and discarded. The 2×2 mm tumor tissue fragments were then placed in sterile, undiluted Matrigel and ice for 60 minutes prior to implantation. (Cancer Res. 55:4670-75, 1995).

### Example 3

### Orthotopic PDAC Model Development

At approximately 8 weeks of age female nu/nu mice were anesthetized with a mixture of ketamine/xylazine given by IM injection. Following establishment of satisfactory anesthesia, a left lateral laparotomy was performed using aseptic technique, and the spleen and pancreas exteriorized by gentle traction. Two of the 2×2 mm² tumor xenograft fragments prepared as described above were anchored to the posterior surface of the splenic portion of the pancreas of each mouse with a 6-0 Prolene suture (Cancer Res. 56:5713-19, 1996; J. Surg. Oncol. 40:261-265, 1989). The abdominal incision was closed with 6-0 Vicryl and the skin closed with skin staples. Mice were observed daily for any signs of infection of the incision line and, following removal of the skin staples, had abdominal palpation performed twice weekly. Establishment of the orthotopic phenotype with diffuse peritoneal spread was accomplished through three serial passes of tumor tissue. Athymic mice with serially passed tumor tissue grafted onto their pancreases exhibited a reproducible diffuse peritoneal metastatic spread from their orthotopically-grafted PDAC tissue.

### Example 4

### Administration of the Compound of Formula (I-a-ii) and Gemcitabine to Mice with Orthotopically Implanted PDAC Tissue and Resulting Enhancement of Survival

Seven days following surgical implantation of serially passed PDAC tumor tissue, mice were randomized into five different treatment groups. Untreated mice (n=5) received no vehicle or drug treatments. The vehicle-treated group (n=10) received 100 microliters of the vehicle used for formulation of the compound of formula (I-a-ii) (40% PEG 1000, 10% povidone, and 2% benzyl alcohol) s.c. (subcutaneously), BID, five days a week. The first monotherapy group (n=10) received the compound of formula (I-a-ii) at 10 mg/kg/dose s.c., BID, five days a week. The second monotherapy group received gemcitabine at 100 mg/kg/dose i.p. (intraperitoneally) twice per week in 0.9% saline as described (Cancer Res. 60: 2926-2935, 2000). The combination therapy group (n=10) received both the compound of formula (I-a-ii) at 10 mg/kg/dose s.c., BID, five days a week, and gemcitabine at 100 mg/kg/dose i.p. twice per week in 0.9% saline as described (Cancer Res. 60: 2926-2935, 2000).

Mice were weighed and palpated twice weekly. Mice remained in the study unless death or overt morbidity occurred. In the latter case, mice were sacrificed. The criteria used for evaluation of overt morbidity were a reduction in body weight of greater than 15%, pronounced hunching and lethargy for several days, the development of ascites that impaired mobility and feeding, or the development of severe jaundice. Upon necropsy, a thorough examination of both the abdominal and thoracic cavities was performed on each mouse in order to determine the extent of gross metastatic spread of the orthotopically-implanted PDAC tumor. Weights of the primary pancreatic tumor with attached spleen, liver and lungs for each mouse were obtained. Primary pancreatic tumor with attached spleen, liver, mesenteric lymph nodes, hepatic lymph nodes and other grossly involved tissues including lungs, kidneys or uterus, were placed in a 10% formalin fixative for histopathologic analysis.

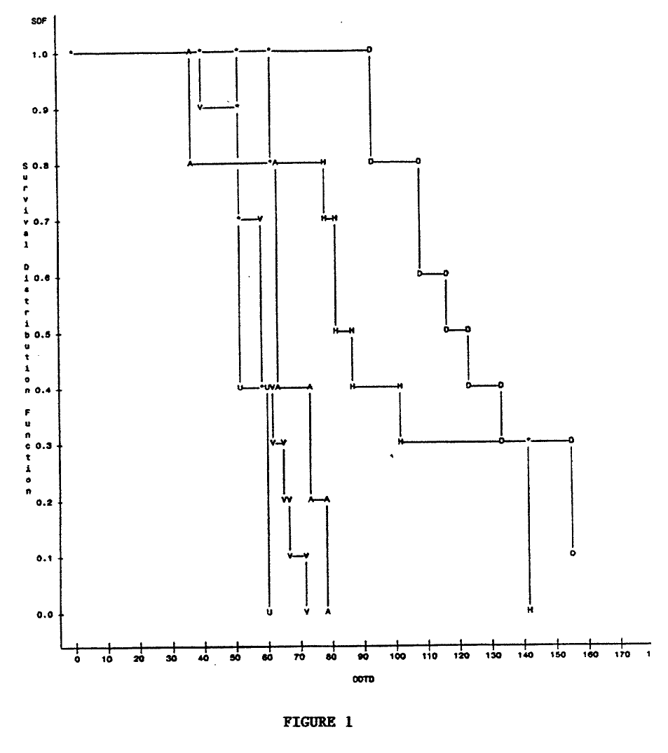

Kaplan-Meier-Wilcoxon analysis of survival data was performed for the days of dosing to death (DDTD) and is shown in FIG. 1. Administration of the compound of formula (I-a-ii) alone resulted in a trend toward improved overall survival relative to administration of controls, but the trend was not significant. The mean survival time for mice who received the compound of formula (I-a-ii), vehicle control, or were untreated, was 63 days, 58 days and 55 days, respectively. The administration of gemcitabine alone prolonged survival relative to the administration of controls. The mean survival time for mice who received gemcitabine, vehicle control, or were untreated, was 97 days, 58 days and 55 days, respectively. The administration of a combination of the compound of formula (I-a-ii) and gemcitabine conferred a highly significant and unexpected improvement in survival relative to administration of gemcitabine alone, the compound of formula (I-a-ii) alone, or controls. The mean survival time of mice who received a combination of the compound of formula (I-a-ii) and gemcitabine, gemcitabine alone, the compound of formula (I-a-ii) alone, vehicle control, or were untreated was 126 days, 97 days, 63 days 58 days, and 55 days, respectively.

### Example 5

### Statistical Analysis

The effects of the compound of formula (I-a-ii) and gemcitabine alone and in combination on survival of tumor-bearing mice were analyzed by the Kaplan-Meier-Wilcoxon method as required for data sets using SAS(SAS 6.12, SAS Institute, Inc. Cary, N.C.). One way ANOVA and Students-Newman-Keuls analyses were used to compare mean survival times between treatment groups. The effects of the compound of formula (I-a-ii) and/or gemcitabine on the weight of primary tumors and incidence of metastatic lesions were assessed by the Dunnet's Multiple comparison test with p<0.05 deemed significant.

Combination therapy using the compositions and methods of the invention has been shown to significantly enhance the survival of nude mice with orthotopically implanted human pancreatic ductal adenocarcinoma. The use of the orthotopic model described above demonstrated a reproducible metastatic phenotype analogous to the human clinical course, with widely disseminated peritoneal, hepatic and messenteric lymph node metastases of human pancreatic carcinoma origin.

As discussed in detail in the foregoing examples, mice with orthotopic human PDAC tumor tissue were untreated or were treated with gemcitabine alone, a trk tyrosine kinase inhibitor (the compound of formula (I-a-ii)) alone, or a combination of gemcitabine and the trk tyrosine kinase inhibitor. The mice who received the combination therapy experienced a significant and unexpected enhancement in survival relative to the mice who received either monotherapy. Moreover, the combination treatment was well-tolerated, with minimal to no toxicity or weight loss observed over a prolonged period of administration. The combination therapy methods and compositions of the present invention have thus been demonstrated to be effective for cancer treatment. Significantly, the efficacy of the combination therapy for enhancing survival exceeds the additive effect of the monotherapies.

### Example 6

### Administration of the Compound of Formula (III-a-i) and Gemcitabine to Mice With Orthotopically Implanted PDAC Tissue and Resulting Enhancement of Survival

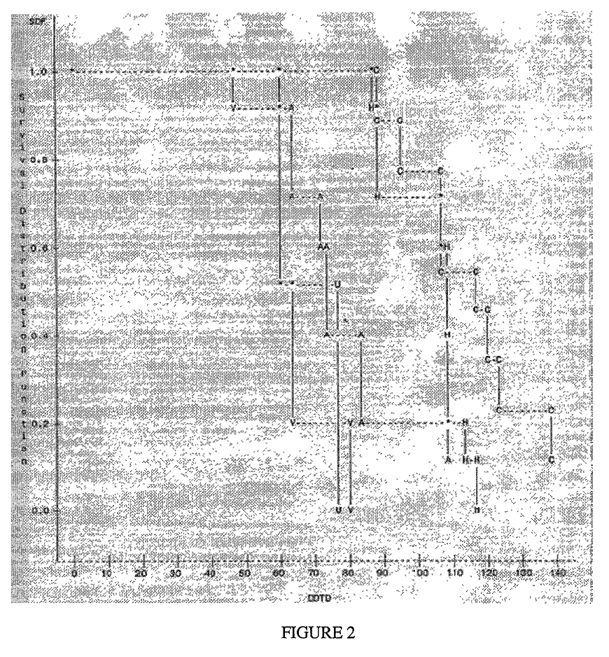

The formulation and administration of the compound of formula (II-a-i) and gemcitabine alone and in combination on survival of tumor-bearing mice was carried out using the methods described in Examples 1, 4, and 5. The data is presented in FIG. 2.

The disclosures of each patent, patent application and publication cited or described in this document are hereby incorporated herein by reference, in their entirety.

Various modifications of the invention, in addition to those described herein, will be apparent to those skilled in the art from the foregoing description. Such modifications are also intended to fall within the scope of the appended claims.

What is claimed is:

1. A method of treating cancer comprising administering to a patient an effective amount of a trk tyrosine kinase inhibitor in combination with an effective amount of an antineoplastic agent.

2. The method of claim 1 wherein the trk tyrosine kinase inhibitor is a compound of the following formula (I): 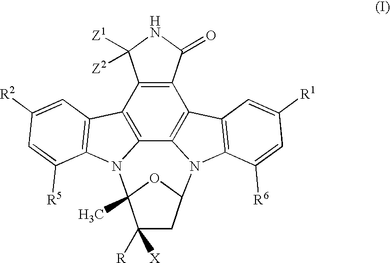
(a) when Z¹ and Z² are both hydrogen:
(1) R is selected from the group consisting of OH, O-n-alkyl of 1-6 carbons, and O-acyl of 2-6 carbons;
(2) X is selected from the group consisting of H; CONHC₆H₅ with the proviso that both R¹and R² are not Br; CH₂Y wherein Y is:
OR⁷ wherein R⁷is H or acyl of 2-5 carbons;
SOR⁸ wherein R⁵ is alkyl of 1-3 carbons, aryl, or a heterocyclic group including a nitrogen atom;
NR⁹R¹⁰ wherein R⁹ and R¹⁰ are each independently H, alkyl of 1-3 carbons, Pro, Ser, Gly, Lys, or acyl of 2-5 carbons, with the proviso that only one of R⁹ and R¹⁰ is Pro, Ser, Gly, Lys or acyl;
SR¹⁶ wherein R¹⁶ is an aryl, alkyl of 1-3 carbons or a heterocyclic group that includes a nitrogen atom;
N₃; CO₂CH₃; S-Glc;
CONR¹¹R¹² wherein R¹¹ and R¹² are each independently H, alkyl of 1-6 carbons, C₆H₅, or hydroxyalkyl of 1-6 carbons, or R¹¹ and R¹² are combined to form —CH₂CH₂OCH₂CH₂—; CO₂CH₃; CH═NNHCONH₂; CONHOH; CH═NOH; 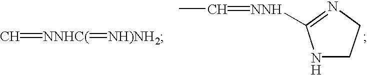
CH═NN(R¹⁷)₂ wherein R¹⁷ represents aryl;
CH₂NHCONBR¹⁸ wherein R¹⁸ is lower alkyl or aryl; or
X and R are combined together to form —CH₂NHCO₂—, —CH₂OC(CH₃)₂O—, ═O, or —CH₂N(CH₃)CO₂—;
(3) each of R¹, R², R⁵ and R⁶ is, independently, H or up to two of them are F; Cl; Br; I; NO₂; CN; OH; NHCONHR¹³ wherein R¹³ is C6Hs or alkyl of 1-3 carbons with the proviso that only one of R¹, R², R⁵ and R⁶ is NHCONHR¹³; CH₂OR¹³; alkyl of 1-3 carbons; CH₂OCONHR¹⁴; or NHCO₂R¹⁴; in which R¹⁴ is lower alkyl; CH(SC₆H₅)₂ or
CH(—SCH₂CH₂S—); or R¹ is CH₂S(O)PR²¹ where p=O or 1, and R²¹ is aryl, alkyl of 1-3 carbons, a heterocyclic group that includes a nitrogen atom, 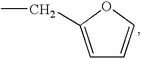
or CH₂CH₂N(CH₃)₂, and R², R⁵, and R⁶ are H; or R¹ is CH═NNR²R²³, wherein R²² and R²³ are each independently H, alkyl of 1-3 carbons, C(═NH)NH₂, or a heterocyclic group that includes a nitrogen atom, or R²² and R²³ are combined together to form —(CH₂)₄—, —(CH₂CH₂OCH₂CH₂)—, or —(CH₂CH₂N(CH₃)CH₂CH₂)—, with the proviso that R²² and R²³ cannot both be H, and at least one of R²² or R²³ is H, except when both are alkyl, and R², R⁵ and R⁶ are H;
and
(b) when Z¹ and Z² are both combined together to represent O; X is CO₂CH₃; R is OH and R¹, R², R⁵ and R⁶ are each hydrogen.

3. The method of claim 2 wherein the compound of formula (I) has the following formula (I-a):

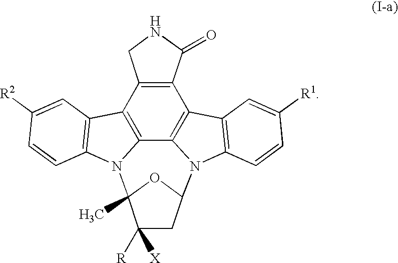

4. The method of claim 3 wherein:
R¹ and R² are selected from H, alkyl, Cl, Br, CH₂OH, CH₂SOCH₂CH₃, NHCONHC₆H₅, CH₂SCH₂CH₃, CH₂SC₆H₅, NHCO₂CH₃, CH₂OC(═O)NHCH₂CH₃, CH═NNH, and CH₂OCH₂CH₃;
R is selected from OH and OCH₃; and
X is selected from OH, CH₂OH, and CO₂alkyl.

5. The method of claim 4 wherein the compound of formula (I-a) is selected from:

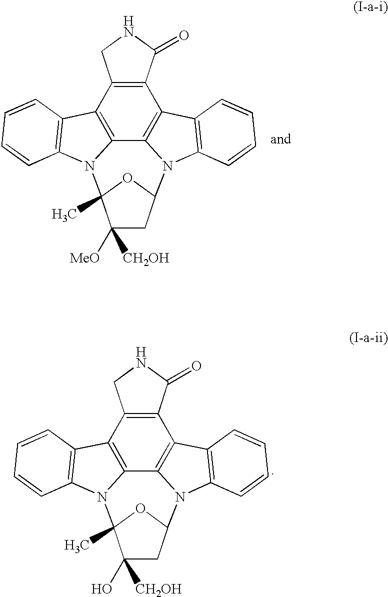

6. The method of claim 5 wherein the compound of formula (a-a) is:

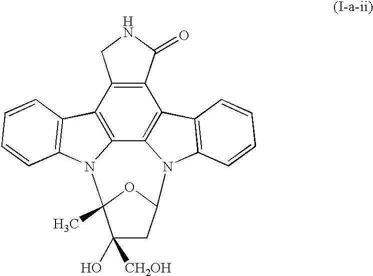

7. The method of claim 1 wherein the antineoplastic agent is selected from the group consisting of fluoropyrimidines, pyrimidine nucleosides and purines.

8. The method of claim 7 wherein the antineoplastic agent is a fluoropyrimidine.

9. The method of claim 8 wherein the fluoropyrimidine is selected from 5-fluorouracil and ftorafur.

10. The method of claim 7 wherein the antineoplastic agent is a pyrimidine nucleoside.

11. The method of claim 10 wherein the pyrimidine nucleoside is selected from the group consisting of gemcitabine, 5-azacytidine, and cytosine arabinoside.

12. The method of claim 11 wherein the pyrimidine nucleoside is gemcitabine.

13. The method of claim 7 wherein the antineoplastic agent is a purine.

14. The method of claim 13 wherein the purine is 6-thioguanine.

15. The method of claim 1 wherein the antineoplastic agent is a compound of the following formula (II): 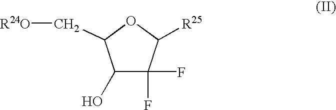
or a pharmaceutically acceptable salt form thereof, wherein:
R²⁴ selected from H and —C(═O)—R²⁸;
R²⁵ is a base defined by one of the following formulae: 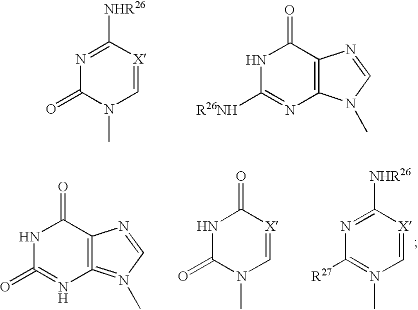
X′ is selected from N and C—R²⁷;
R²⁶ is selected from H, alkyl and —C(═O)—R²⁸;
R²⁷ is selected from H, alkyl, amino, bromo, fluoro, chloro and iodo; and
R²⁸ is selected from H and alkyl;
and a pharmaceutically acceptable carrier.

16. The method of claim 15 wherein R²⁵ is a base of the following formula

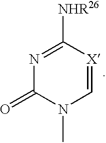

17. The method of claim 16 wherein X′ is C—R²⁷.

18. The method of claim 17 wherein R²⁴, R²⁶, R²⁷ and R²⁸ are H.

19. The method of claim 1 wherein the trk tyrosine kinase inhibitor is a compound of the following formula 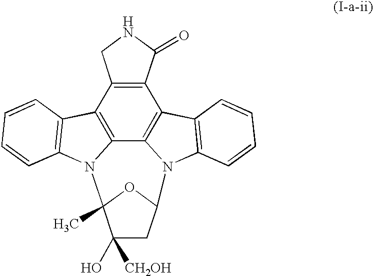
and the antineoplastic agent is a compound of the following formula

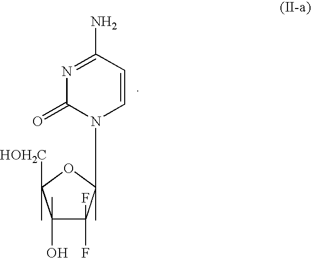

20. The method of claim 19 wherein the cancer comprises pancreatic cancer.

21. The method of claim 20 wherein the pancreatic cancer is pancreatic ductal adenocarcinoma.

22. The method of claim 20 wherein the compound of formula (II-a) is administered to the patient in a substantial excess relative to the compound of formula (I-a-ii).

23. The method of claim 22 wherein the compound of formula (II-a) and the compound of formula (I-a-ii) are administered to the patient in a weight ratio of from about 4:1 to about 20:1.

24. The method of claim 1 wherein the cancer is selected from the group consisting of carcinomas of the pancreas, prostate, breast, thyroid, colon, and lung; malignant melanomas; glioblastomas; neuroectodermal-derived tumors; and leukemias.

25. The method of claim 1 wherein the cancer is pancreatic cancer.

26. The method of claim 25 wherein the pancreatic cancer is pancreatic ductal adenocarcinoma.

27. A pharmaceutical composition comprising an effective amount of a trk tyrosine kinase inhibitor and an effective amount of an antineoplastic agent, together with a pharmaceutically acceptable carrier.

28. The composition of claim 27 wherein the trk tyrosine kinase inhibitor is a compound of the following formula (I): 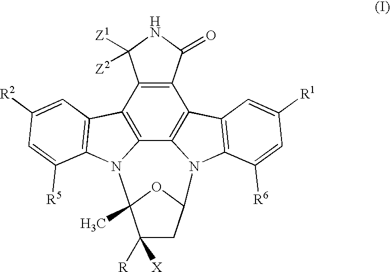
or a stereoisomer or pharmaceutically acceptable salt form thereof, wherein:
(a) when Z¹ and Z² are both hydrogen:
(1) R is selected from the group consisting of OH, O-n-alkyl of 1-6 carbons, and O-acyl of 2-6 carbons;
(2) X is selected from the group consisting of H; CONHC₆H₅ with the proviso that both R¹and R² are not Br; CH₂Y wherein Y is:
OR⁷ wherein R⁷ is H or acyl of 2-5 carbons;
SOR⁸wherein R⁸ is alkyl of 1-3 carbons, aryl, or a heterocyclic group including a nitrogen atom;
NR⁹R¹⁰ wherein R⁹ and R¹ are each independently H, alkyl of 1-3 carbons, Pro, Ser, Gly, Lys, or acyl of 2-carbons, with the proviso that only one of R⁹ and R¹⁰ is Pro, Ser, Gly, Lys or acyl;
SR¹⁶ wherein R¹ is an aryl, alkyl of 1-3 carbons or a heterocyclic group that includes a nitrogen atom;
N₃; CO₂CH₃; S-Glc;
CONR¹¹R¹² wherein R¹¹ and R¹² are each independently H, alkyl of 1-6 carbons, C₆H₅, or hydroxyalkyl of 1-6 carbons, or R¹¹ and R¹² are combined to form —CH₂CH₂OCH₂CH₂—;
CO₂CH₃; CH═NNHCONH₂; CONHOH; CH═NOH; CH═NNHC(═NH)NH₂; 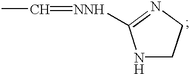
CH═NN(R¹⁷)₂ wherein R¹⁷ represents aryl;
CH₂NHCONHR¹⁸ wherein R¹⁸ is lower alkyl or aryl; or
X and R are combined together to form —CH₂NHCO₂—, —CH₂OC(CH₃)₂O—, =0, or —CH₂N(CH₃)CO₂—;
(3) each of R¹, R², R⁵ and R⁶ is, independently, H or up to two of them are F; Cl; Br; I; NO₂; CN; OH; NHCONHR wherein R is C₆H₅ or alkyl of 1-3 carbons with the proviso that only one of R¹, R², R⁵ and R⁶ is NHCONHR¹³; CH₂OR³; alkyl of 1-3 carbons; CH₂OCONHR¹⁴; or NHCO₂R¹⁴; in which R¹⁴ is lower alkyl; CH(SC₆H₅)₂ or CH(—SCH₂CH₂S—); or R¹ is CH₂S(O)ₚR²¹ where p=O or 1, and R²¹ is aryl, alkyl of 1-3 carbons, a heterocyclic group that includes a nitrogen atom, 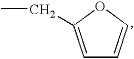
or CH₂CH₂N(CH₃)₂, and R², R⁵, and R⁶ are H; or R¹ is CH═NNR²²R²³, wherein R²² and R²³ are each independently H, alkyl of 1-3 carbons, C(═NH)NH₂, or a heterocyclic group that includes a nitrogen atom, or R 22 and R²³ are combined together to form —(CH₂)₄—, —(CH₂CH₂OCH₂CH₂)—, or —(CH₂CH₂N(CH₃)CH₂CH₂)—, with the proviso that R²² and R²³ cannot both be H, and at least one of R²² or R²³ is H, except when both are alkyl, and R², R⁵ and R⁶ are H;
and
(b) when Z¹ and Z² are both combined together to represent O; X is CO₂CH₃; R is OH and R¹, R², R⁵ and R⁶ are each hydrogen.

29. The composition of claim 28 wherein the compound of formula (I) has the following formula (I-a):

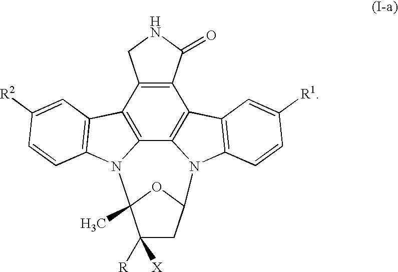

30. The composition of claim 29 wherein:
R¹ and R² are selected from H, alkyl, Cl, Br, CH₂OH, CH₂SOCH₂CH₃, NHCONHC₆H₅, CH₂SCH₂CH₃, CH₂SC₆H₅, NHCO₂CH₃, CH₂OC(═O)NHCH₂CH₃, CH═NNH, and CH₂OCH₂CH₃;
R is selected from OH and OCH₃; and
X is selected from OH, CH₂OH, and CO₂alkyl.

31. The composition of claim 30 wherein the compound of formula (I-a) is selected from:

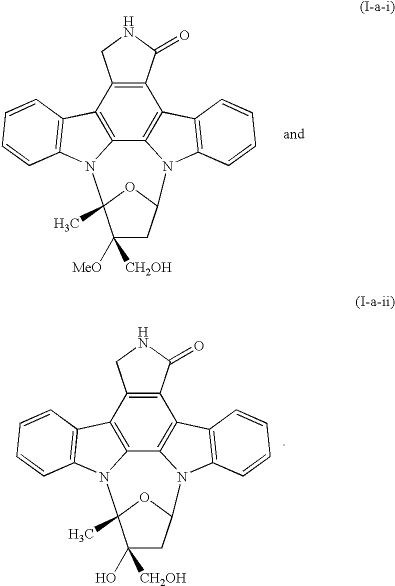

32. The composition of claim 31 wherein the compound of formula (I-a) is:

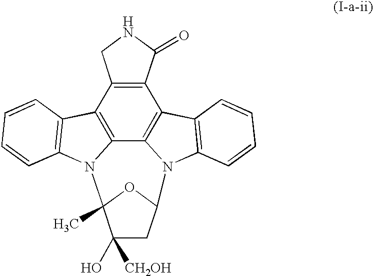

33. The composition of claim 27 wherein the antineoplastic agent is selected from the group consisting of fluoropyrimidines, pyrimidine nucleosides and purines.

34. The composition of claim 33 wherein the antineoplastic agent is a fluoropyrimidine.

35. The composition of claim 34 wherein the fluoropyrimidine is selected from the group consisting of 5-fluorouracil and ftorafur.

36. The composition of claim 33 wherein the antineoplastic agent is a pyrimidine nucleoside.

37. The composition of claim 36 wherein the pyrimidine nucleoside is selected from the group consisting of gemcitabine, 5-azacytidine, and cytosine arabinoside.

38. The composition of claim 37 wherein the pyrimidine nucleoside is gemcitabine.

39. The composition of claim 33 wherein the antineoplastic agent is a purine.

40. The composition of claim 39 wherein the purine is 6-thioguanine.

41. The composition of claim 27 wherein the antineoplastic agent is a compound of the following formula (IL): 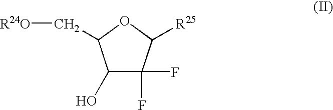
or a pharmaceutically acceptable salt form thereof, wherein:
R²⁴ is selected from H and —C(═O)—R²¹;
R²⁵ is a base defined by one of the following formulae: 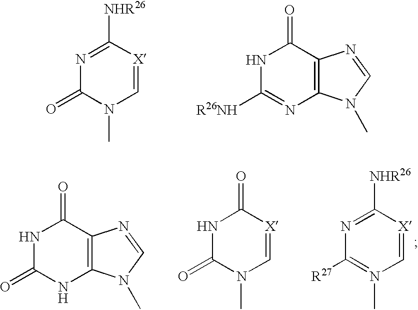
X′ is selected from N and C—R²⁷;
R²⁶ is selected from H, alkyl and —C(═O)—R
R²⁷ is selected from H, alkyl, amino, bromo, fluoro, chloro and iodo; and
R²⁸ is selected from H and alkyl;
and a pharmaceutically acceptable carrier.

42. The composition of claim 41 wherein R²⁵ is a base of the following formula

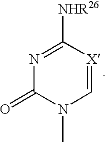

43. The composition of claim 42 wherein X′ is C—R²⁷.

44. The composition of claim 43 wherein R²⁴, R²⁶, R²⁷ and R²⁸ are H.

45. The composition of claim 27 wherein the trk tyrosine kinase inhibitor is a compound of the following formula: 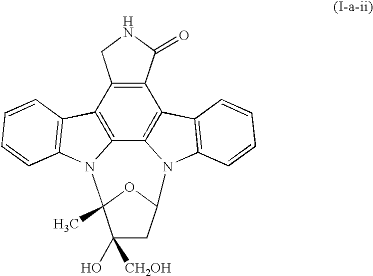
and the antineoplastic agent is a compound of the following formula:

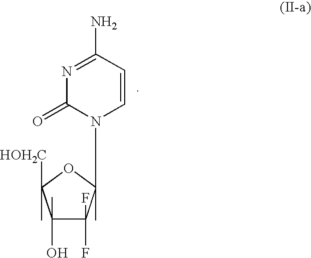

46. The composition of claim 45 wherein the compound of formula (II-a) is present in the composition in a substantial excess relative to the compound of formula (I-a-ii).

47. The composition of claim 46 wherein the compound of formula (II-a) and the compound of formula (I-a-ii) are present in the composition in a weight ratio of from about 4:1 to about 20:1.

48. The composition of claim 27 which is in a single dosage unit form.

49. A pharmaceutical kit comprising one or more containers containing pharmaceutical dosage units comprising an effective amount of a trk tyrosine kinase inhibitor in combination with an effective amount of an antineoplastic agent.

50. The kit of claim 49 further comprising conventional pharmaceutical kit components.

51. The method of claim 1 wherein the trk tyrosine kinase inhibitor is a compound of the following formula (III): 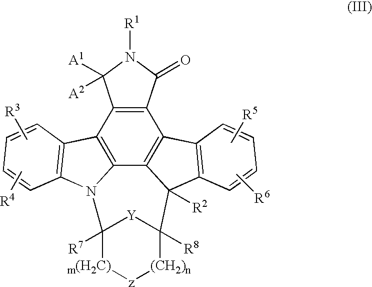
or a stereoisomer or pharmaceutically acceptable salt form thereof, wherein:
R¹ is selected from the group consisting of:
a) H, substituted or unsubstituted C₁₋₄ alkyl, substituted or unsubstituted aryl, substituted or unsubstituted arylalkyl, substituted or unsubstituted heteroaryl, or substituted or unsubstituted heteroarylalkyl;
b) —C(═O)R⁹, where R⁹ is selected from the group consisting of H, C₁₋₄ alkyl, aryl and heteroaryl;
c) —OR¹⁰, where R¹⁰ is selected from the group consisting of H and C₁₋₄ alkyl;
d) —C(═O)NH₂, —NR¹¹R¹²-(CH₂)ₚNR¹¹R¹², —(CH₂)ₚOR¹⁰, —O(CH₂)ₚOR¹⁰ and —O(CH₂)ₚNR¹¹R¹², wherein p is from 1 to 4; and wherein either
1) R¹¹ and R¹² are each independently selected from the group consisting of H and C₁₋₄ alkyl; or
2) R¹¹ and R¹² together form a linking group of the formula —(CH₂)₂—X¹—(CH₂)₂—, wherein X¹ is selected from the group consisting of —O—, —S—, and —CH₂—;
R² is selected from the group consisting of H, C₁₋₄ alkyl, —OH, and C₁₋₄ alkoxy;
R³, R⁴, R⁵ and R⁶ are each independently selected from the group consisting of:
a) H, aryl, heteroaryl, F, Cl, Br, I, —CN, CF₃, —NO₂, —OH, —OR⁹, —O(CH₂)ₚNR¹¹R¹², —OC(═O)R⁹, —OC(═O)NR²R⁷, —OC(═O)NR¹¹R¹², —O(CH₂)ₚOR¹¹, —CH₂OR¹⁰, NR¹¹R¹², —NR¹⁰S(═O)₂R⁹, NR¹⁰C(═O)R⁹;
b) —CH₂OR¹⁴, wherein R¹⁴ is the residue of an amino acid after the hydroxyl group of the carboxyl group is removed;
c) —NR¹⁰C(═O)NR¹¹R¹², —CO₂R², —C(═O)R², —C(═O)NR¹¹R¹², —CH═NOR², —CH═NR⁹, —(CH₂)ₚNR¹¹R¹², —(CH₂)ₚNHR¹⁴, or —CH═NNR²R^{2A} wherein R^{2A} is the same as R²;
d) —S(O)_{y}R²—(CH₂)ₚS(O)_{y}R⁹, —CH₂S(O)_{y}R¹⁴ wherein y is 0, 1 or 2;
e) C₁₋₈ alkyl, C₂₋₈ alkenyl, C₂₋₈ alkynyl, wherein each alkyl, alkenyl, or alkynyl group may substituted with 1 to 3 groups selected from the group consisting of:
C₆₋₁₀ arylalkyl, heteroaryl, arylalkoxy, heterocycloalkoxy, hydroxyalkoxy, alkyloxy-alkoxy, hydroxyalkylthio, alkoxyalkylthio, F, Cl, Br, I, —CN, —NO₂, —OH, —OR⁹, —X²(CH₂)ₚNR¹¹R¹², —X²(CH₂)ₚC(═O)NR¹¹R¹², —X²(CH₂)ₚOC(═O)NR¹¹R¹², —X²(CH₂)ₚCO₂R⁹, —X² (CH₂)ₚS(O)^{y}R⁹, —X²(CH₂)ₚNR¹⁰C(═O)NR¹¹R¹², —OC(═O)R⁹, —OCONHR², —O-tetrahydropyranyl, —NR¹¹R¹², —NR¹⁰C(═O)R⁹, —NR¹⁰CO₂R⁹, —NR¹⁰C(═O)NR¹¹R¹², —NHC(═NH)NH₂, NR¹⁰S(O)₂R⁹, —S(O)_{y}R⁹, —
CO₂R², —C(═O)NR¹¹R², —C(═O)R², —CH₂OR¹⁰, —CH═NNR²R^{2A}, —CH═NOR², —CH═NR⁹, —CH═NNHCH(N═NH)NH₂, —S(═O)₂NR²R^{2A}, —P(═O)(OR¹⁰)₂, —OR¹⁴, and a monosaccharide having from 5 to 7 carbons wherein each hydroxyl group of the monosaccharide is independently either unsubstituted or is replaced by H, C₁₋₄ alkyl, alkylcarbonyloxy having from 2 to 5 carbons, or C₁₋₄ alkoxy;
wherein X² is O, S, or NR¹⁰;
R⁷ and R⁸ are each independently selected from the group consisting of H, C₁₋₄ alkyl, C₁₋₄ alkoxy, substituted or unsubstituted C₆₋₁₀ arylalkyl, substituted or unsubstituted heteroarylalkyl, —(CH₂)ₚOR¹⁰, —(CH₂)ₚOC(═O)NR¹¹R¹², and —(CH₂)ₚNR¹¹R¹²;
or R⁷ and R⁸ together form a linking group of the formula —CH₂—X³—CH₂—; wherein X³ is X² or a bond;
m and n are each independently 0, 1, or 2;
Y is selected from the group consisting of —O—, —S—, —N(R¹⁰)—, —N+(O—)(R¹⁰)—, —N(OR¹⁰)—, and —CH₂—;
Z is selected from the group consisting of a bond, —O—, —CH═CH—, —S—, —C(═O)—, —CH(ORO)—, —N(RO)—, —N(OR¹⁰)—, CH(NR¹¹R¹²)—, —C(═O)N(R¹⁷), N(R¹⁷)C(═O)—, —N(S(O)_{y}R⁹)—, —N(S(O)_{y}NR¹¹R¹²)—, —N(C(═O)R¹⁷)—, —C(R⁵ R¹⁶)—, —N⁺(O⁻)(R¹⁰)—, —CH(OH)—CH(OH)—, and —CH(O(C═O)R )CH(OC(═O)R^{9A})—;
wherein
R^{9A} is the same as R⁹;
R¹⁵ and R¹⁶ are independently selected from the group consisting of H, —OH, —C(═O)R¹⁰, —O(C═O)R⁹, hydroxyalkyl, and —CO₂R¹⁰;
R¹⁷ is selected from the group consisting of H, C₁₋₆ alkyl, aryl, and heteroaryl;
A¹ and A² are selected from the group consisting of H, H; H, OR²; H, —SR²; H, —N(R²)₂; and a group wherein A¹ and A² together form a moiety selected from the group consisting of ═O, ═S, and ═NR².

52. The method of claim 51 wherein the compound of formula (III) has the following formula (III-a): 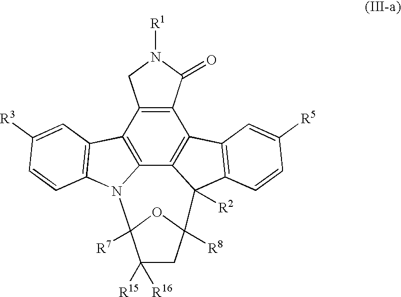
wherein:
R¹ is H or substituted or unsubstituted C₁₋₄ alkyl;
R⁷ and R⁸ are each independently H or substituted or unsubstituted C₁₋₄ alkyl.

53. The method of claim 52 wherein:
R¹ is H;
R³ and R⁵ are each independently H, aryl, F, Cl, Br, I, —OR⁹, C₁₋₈ alkyl, C₂₋₈ alkenyl, or C₂₋₈ alkynyl.

54. The method of claim 53 wherein the compound of formula (III-a) is:

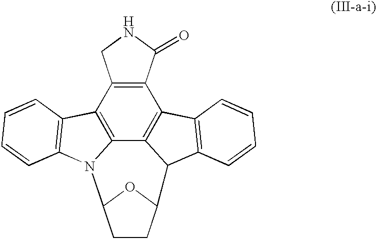

55. The method of claim 1 wherein the trk tyrosine kinase inhibitor is a compound of the following formula: 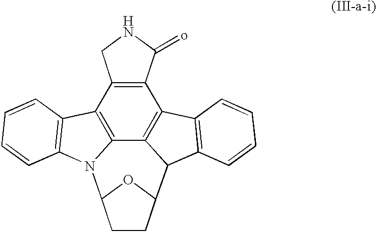
and the antineoplastic agent is a compound of the following formula:

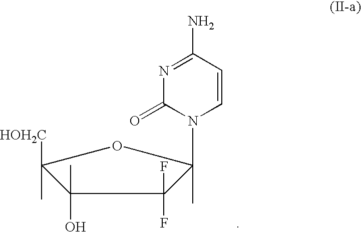

56. The method of claim 55 wherein the cancer comprises pancreatic cancer.

57. The method of claim 56 wherein the pancreatic cancer is pancreatic ductal adenocarcinoma.

58. The method of claim 56 wherein the compound of formula (II-a) is administered to the patient in a substantial excess relative to the compound of formula (III-a-i).

59. The method of claim 58 wherein the compound of formula (II-a) and the compound of formula (III-a-i) are administered to the patient in a weight ratio of from about 4:1 to about 20:1.

60. The composition of claim 27 wherein the trk tyrosine kinase inhibitor is a compound of the following formula (III): 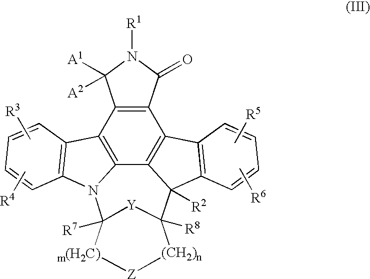
or a stereoisomer or pharmaceutically acceptable salt form thereof, wherein:
R¹ is selected from the group consisting of:
a) H, substituted or unsubstituted C₁₋₄ alkyl, substituted or unsubstituted aryl, substituted or unsubstituted arylalkyl, substituted or unsubstituted heteroaryl, or substituted or unsubstituted heteroarylalkyl;
b) —C(═O)R⁹, where R⁹ is selected from the group consisting of H, C₁₋₄ alkyl, aryl and heteroaryl;
c) —OR¹⁰, where R¹⁰ is selected from the group consisting of H and C₁₋₄ alkyl;
d) —C(═O)NH₂, —NR¹¹R¹², —(CH₂)ₚNR¹¹R¹², —(CH₂)ₚOR¹⁰, —O(CH₂)ₚOR¹⁰ and —O(CH₂)ₚNR¹¹R², wherein p is from 1 to 4; and wherein either
1) R¹¹ and R¹² are each independently selected from the group consisting of H and C₁₋₄ alkyl; or
2) R¹¹ and R¹² together form a linking group of the formula —(CH₂)₂—X¹—(CH₂)₂—, wherein X¹ is selected from the group consisting of —O—, —S—, and —CH₂—;
R² is selected from the group consisting of H, C₁₋₄ alkyl, —OH, and C₁₋₄ alkoxy;
R³, R⁴, R⁵ and R⁶ are each independently selected from the group consisting of:
a) H, aryl, heteroaryl, F, Cl, Br, I, —CN, CF₃, —NO₂, —OH, —OR⁹, —O(CH₂)ₚNR¹¹R¹², —OC(═O)R⁹, —OC(═O)NR R⁷—OC(═O)NR¹¹R¹², —O(CH₂)ₚOR¹⁰, —CH₂OR¹⁰, —NR¹¹R¹²—NR¹⁰S(═O)₂R⁹, —NR¹⁰C(═O)R⁹;
b) —CH₂OR 14, wherein R¹⁴ is the residue of an amino acid after the hydroxyl group of the carboxyl group is removed;
c) —NR¹⁰C(═O)NR¹¹R¹², —CO₂R², —C(═O)R², —C(═O)NR¹¹R¹², —CH═NOR², —CH═NR⁹, —(CH₂)ₚNR¹¹R¹², —(CH₂)ₚNHR⁴, or CH═NNR²R^{2A} wherein R²A is the same as R
d) _S(O)R²—(CH₂)ₚS(O),R⁹, —CH₂S(O)_{y}R¹⁴ wherein y is 0, 1 or 2;
e) C₁₋₈ alkyl, C₂₋₈ alkenyl, C₂₋₈ alkynyl, wherein each alkyl, alkenyl, or alkynyl group may substituted with 1 to 3 groups selected from the group consisting of:
C₆₋₁₀ arylalkyl, heteroaryl, arylalkoxy, heterocycloalkoxy, hydroxyalkoxy, alkyloxy-alkoxy, hydroxyalkylthio, alkoxyalkylthio, F, Cl, Br, I, —CN, —NO₂, —OH, —OR⁹, —X²(CH₂)ₚNR¹¹R¹², —X²(CH₂)ₚC(═O)NR¹¹R¹², —X²(CH₂),OC(═O)NR¹R¹², —X²(CH₂)ₚCO₂R⁹, —X²(CH₂)ₚS(O)_{y}R⁹, —X²(CH₂)ₚNR¹⁰C(═O)NR¹¹R¹², —OC(═O)R⁹, —OCONHR², —O-tetrahydropyranyl, —NR¹¹R¹², —NR¹⁰C(═O)R⁹, —NR¹⁰CO₂R⁹, —NR¹⁰C(═O)NR¹¹R¹², —NHC(═NH)NH₂, NR¹⁰S(O)₂R⁹, —S(O)_{y}R⁹—CO₂R², —C(═O)NR¹¹R¹², —C(═O)R², —CH₂OR¹⁰, —CH═NNR²R^{2A}, —CH═NOR², —CH═NR⁹, —CH═NNHCH(N═NH)NH₂, —S(═O)₂NR²R^{2A}, —P(═O)(OR¹⁰)₂, —OR¹⁴, and a monosaccharide having from 5 to 7 carbons wherein each hydroxyl group of the monosaccharide is independently either unsubstituted or is replaced by H, C₁₋₄ alkyl, alkylcarbonyloxy having from 2 to 5 carbons, or C₁₋₄ alkoxy;
wherein X² is O, S, or NR¹⁰;
R⁷ and R⁸ are each independently selected from the group consisting of H, C₁₋₄ alkyl, C₁₋₄ alkoxy, substituted or unsubstituted C₆₋₁₀ arylalkyl, substituted or unsubstituted heteroarylalkyl, —(CH₂)ₚOR¹⁰, —(CH₂)ₚOC(═O)NR¹¹R¹², and —(CH₂)ₚNR¹¹R¹²;
or R⁷ and R⁸ together form a linking group of the formula —CH₂—X³—CH₂—; wherein X³ is X² or a bond;
m and n are each independently 0, 1, or 2;
Y is selected from the group consisting of —O—, —S—, —N(R¹⁰)—, —N⁺(O⁻)(R¹⁰)—, —N(OR¹⁰)—, and —CH₂—;
Z is selected from the group consisting of a bond, —O—, —CH═CH—, —S—, —C(═O)—, —CH(OR¹⁰), —N(R¹⁰)—, —N(OR¹⁰)—, CH(NR¹¹R¹²) —C(═O)N(R¹⁰) N(R⁷)C(═O)—, —N(S(O)_{y}R⁹)—, —N(S(O)_{y}NR¹¹R¹²)—, —N(C(═O)R¹⁷)—, —C(R¹⁵R¹⁶), —N⁺(O⁻)(R¹⁰)—, —CH(OH)—CH(OH)—, and —CH(O(C═O)R⁹)CH(OC(═O)R^{9A})—;
wherein
R^{9A} is the same as R⁹;
R¹⁵ and R¹⁶ are independently selected from the group consisting of H, —OH, —C(═O)R¹⁰, —O(C═O)R⁹, hydroxyalkyl, and —CO₂R¹⁰;
R¹⁷ is selected from the group consisting of H, C₁₋₆ alkyl, aryl, and heteroaryl;
A¹ and A² are selected from the group consisting of H, H; H, OR²; H, —SR²; H, —N(R²)₂; and a group wherein A¹ and A² together form a moiety selected from the group consisting of ═O, ═S, and ═NR².

61. The composition of claim 60 wherein the compound of formula (1R) has the following formula (III-a): 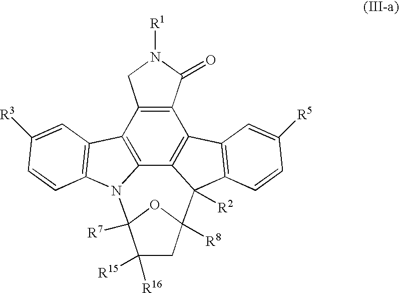
wherein:
R¹ is H or substituted or unsubstituted C₁₋₄ alkyl;
R⁷ and R⁸ are each independently H or substituted or unsubstituted C₁₋₄ alkyl.

62. The composition of claim 61 wherein:
R¹ is H;
R³ and R⁵ are each independently H, aryl, F, Cl, Br, I, —OR⁹, C₁₋₈ alkyl, C₂₋₈ alkenyl, or C₂₋₈ alkynyl.

63. The composition of claim 62 wherein the compound of formula (III-a) is:

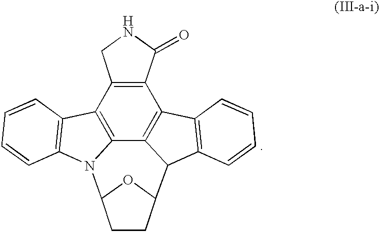

64. The composition of claim 27 wherein the trk tyrosine kinase inhibitor is a compound of the following formula 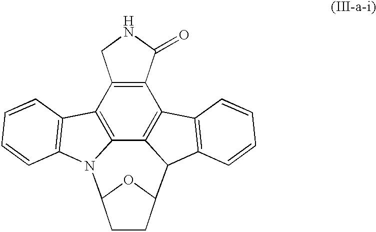
and the antineoplastic agent is a compound of the following formula

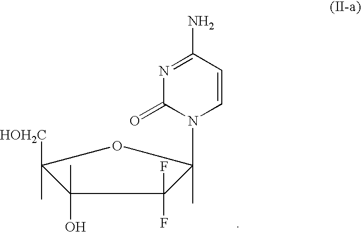

65. The composition of claim 64 wherein the compound of formula (III-a) is present in the composition in a substantial excess relative to the compound of formula (III-a-i).

66. The composition of claim 65 wherein the compound of formula (III-a) and the compound of formula (III-a-i) are present in the composition in a weight ratio of from about 4:1 to about 20:1.
